(19) United States
(12) Patent Application Publication
(10) Pub. No.: US 2016/0302978 A1
(43) Pub. Date: Oct. 20, 2016

(54) MULTI-LINER DISPOSABLE ABSORBENT PRODUCT

(71) Applicant: SCA HGIENE PRODUCTS AB, Göteborg (SE)

(72) Inventor: Åsa LINDSTRÖM, Göteborg (SE)

(21) Appl. No.: 15/102,875

(22) PCT Filed: Dec. 16, 2013
(86) PCT No.: PCT/SE2013/051523

§ 371 Date: Jun. 9, 2016

(51) Int. Cl. A61F 13/505; A61F 13/56; A61F 13/62; A61F 13/47

(52) U.S. Cl. CPC A61F 13/505; A61F 13/4704; A61F 13/5616; A61F 13/625; A61F 2013/4708

(57) ABSTRACT

A disposable absorbent product has a longitudinal dimension and a transverse dimension orthogonal to the longitudinal dimension. The product has a first absorbent assembly, as well as a second absorbent assembly that overlays the first absorbent assembly. The first absorbent assembly includes a first topsheet and a fluid-impervious first backsheet in overlaying relationship with one another, with the first backsheet and the first topsheet being respectively configured to face away from and toward a wearer of the disposable absorbent product during use. The first absorbent assembly has a pair of lateral edges. The second absorbent assembly is releasably coupled to the first absorbent assembly, and includes a second topsheet configured to face toward the wearer of the absorbent product during use, and a second backsheet adjacent the first topsheet and in overlaying relationship with the second topsheet.

### CROSS-REFERENCE TO PRIOR APPLICATION

This application is a §371 National Stage Application of PCT International Application No. PCT/SE2013/051523 filed Dec. 16, 2013, which is incorporated herein in its entirety.

### TECHNICAL FIELD

The present disclosure is generally related to absorbent products and, more particularly, to disposable absorbent products made up of more than one absorbent liner and which are worn by humans for the containment and absorption of fluid bodily secretions.

### BACKGROUND

Disposable absorbent products for absorption of bodily fluids are available in different types, designs, and dimensions. For example, training pants, baby diapers, adult diapers, and incontinence guards are products designed for the containment of urine and excrement. There are other types of disposable absorbent articles, such as feminine hygiene products (e.g., heavy and light incontinence pads, pantiliners) that are primarily designed to retain urine and/or menses secreted by female wearers.

One known type of disposable absorbent product takes the form of a double-liner product. A known double-liner product includes two or more individual absorbent liners, each individually capable of retaining fluids secreted by the wearer, and which are coupled in a manner that permits their separation, particularly after one of the liners has been soiled. In products of that type, it has been observed that only a portion of the liner in direct contact with the skin is typically soiled, making the presence of other regions of the product unnecessary, wasteful, and or detrimental to the wearer's comfort.

It is therefore desirable to provide disposable absorbent products of the type generally described above, that address these and other shortcomings.

### SUMMARY

In one embodiment, a disposable absorbent product is provided having a longitudinal dimension and a transverse dimension orthogonal to the longitudinal dimension. The product has a first absorbent assembly, as well as a second absorbent assembly that overlays the first absorbent assembly. The first absorbent assembly includes a first topsheet and a fluid-impervious first backsheet in overlaying relationship with one another, with the first backsheet and the first topsheet being respectively configured to face away from and toward a wearer of the disposable absorbent product during use. The first absorbent assembly has a pair of lateral edges.

The second absorbent assembly is releasably coupled to the first absorbent assembly, and includes a second topsheet configured to face toward the wearer of the absorbent product during use, and a second backsheet adjacent the first topsheet and in overlaying relationship with the second topsheet. The second absorbent assembly has a central portion and a pair of opposed lateral regions protruding transversely from the central portion so as to define a pair of foldable wings of the product. The lateral regions of the second absorbent assembly have respective opposed outer lateral edges, each located distally from an adjacent one of the lateral edges of the first absorbent assembly.

The product may further include a first fastener on an exposed surface of the first backsheet, adapted to secure the product to a garment worn by the wearer of the product, and a second fastener in the lateral regions of the second absorbent assembly, also adapted to secure the product to the garment worn by the wearer of the product. One or both of the fasteners may include an adhesive. Additionally or alternatively, at least one of the first fastener or the second fastener may include a plurality of hooks adapted for entangled engagement with the garment worn by the wearer of the product. The second fastener may include a pair of opposed adhesive elements, each inboard of a respective adjacent one of the outer lateral edges at the lateral regions. In specific embodiments, the second fastener is disposed on the second backsheet.

The lateral regions may be foldable along respective fold lines, with each fold line defining a border between the central portion and the lateral regions of the second absorbent assembly. In specific embodiments, the fold lines are defined by the lateral edges of the first absorbent assembly when the lateral regions are folded. The first and second absorbent assemblies may be releasably coupled to one another through a plurality of bonds located in the central portion of the second absorbent assembly. Additionally, the bonds may extend through the second topsheet of the second absorbent assembly. In specific embodiments, the first topsheet is fluid pervious and the second backsheet is fluid impervious. The product may further include an absorbent core in at least one of the first absorbent assembly or the second absorbent assembly, and disposed between the first topsheet and the first backsheet, or between the second topsheet and the second backsheet, respectively.

In another embodiment, a disposable absorbent product is provided that has a longitudinal dimension and a transverse dimension orthogonal to the longitudinal dimension. The disposable absorbent product has a first absorbent assembly and a second absorbent assembly that overlays the first absorbent assembly. The first absorbent assembly includes a first topsheet and a fluid-impervious first backsheet in overlaying relationship with one another, with the first backsheet and the first topsheet being respectively configured to face away from and toward a wearer of the product during use. The first absorbent assembly has a pair of lateral edges. The second absorbent assembly is releasably coupled to the first absorbent assembly, and includes a second topsheet configured to face toward the wearer of the product during use and a second backsheet adjacent the first topsheet and in overlaying relationship with the second topsheet.

The second absorbent assembly has a central portion and a pair of opposed lateral regions protruding transversely from the central portion so as to define a pair of foldable wings of the product. A first adhesive is disposed on an exposed surface of the first backsheet, and is adapted to secure the product to a garment worn by the wearer of the product. A second adhesive is disposed on the second backsheet, in the lateral regions of the second absorbent assembly, and is also adapted to secure the product to the garment worn by the wearer of the product. The lateral regions of the second absorbent assembly have respective opposed outer lateral edges, with each of the outer lateral edges being located distally from an adjacent one of the lateral edges of the first absorbent assembly.

In yet another embodiment, a disposable absorbent product is provided having a longitudinal dimension and a transverse dimension orthogonal to the longitudinal dimension. The product includes a first absorbent assembly having a first topsheet and a fluid-impervious first backsheet in overlaying relationship with one another, with the first backsheet and the first topsheet being respectively configured to face away from and toward a wearer of the product during use. A second absorbent assembly overlays the first absorbent assembly, and is releasably coupled to the first absorbent assembly. The second absorbent assembly includes a second topsheet configured to face toward the wearer of the product during use, and a second backsheet adjacent the first topsheet and in overlaying relationship with the second topsheet. The second absorbent assembly has a length no greater than about 75% of a length of the first absorbent assembly.

The second absorbent assembly in specific embodiments has a length that is no greater than about 50% of the length of the first absorbent assembly. The second absorbent assembly may have a central portion and a pair of opposed lateral regions protruding transversely from the central portion so as to define a pair of foldable wings of the product. Additionally, the first absorbent assembly may have a central portion and a pair of opposed lateral regions protruding transversely from the central portion of the first absorbent assembly, so as to further define the pair of foldable wings of the product. The central portion of the second absorbent assembly may be longitudinally coextensive with the lateral regions of that second absorbent assembly. Additionally or alternatively, the central portion of the second absorbent assembly may have a pair of longitudinally opposed end edges that are substantially linear, or may be such that at least one of the end edges is arcuate.

### BRIEF DESCRIPTION OF THE DRAWINGS

The objectives and features of the invention will become more readily apparent from the following detailed description taken in conjunction with the accompanying drawings in which:

FIG. 1 is a perspective view of a disposable absorbent product in accordance with one embodiment of the invention.

FIG. 2 is a partially disassembled, perspective view of the disposable absorbent product of FIG. 1.

FIG. 3 is a schematic, cross-sectional view taken generally along line 3-3 of FIG. 1.

FIG. 4 is a view similar to FIG. 3, illustrating a disposable absorbent product in accordance with another embodiment of the invention.

FIG. 4A is a view similar to FIGS. 3 and 4, illustrating a disposable absorbent product in accordance with yet another embodiment of the invention.

FIG. 5 is a perspective view of one of the disposable absorbent products of any of FIG. 1-4 or 4A, illustrating an exemplary separation of components thereof.

FIG. 6 is a perspective view of a disposable absorbent product in accordance with another embodiment of the invention.

FIG. 7 is a partially disassembled, perspective view of the disposable absorbent product of FIG. 6.

FIG. 8 is a schematic, cross-sectional view taken generally along line 8-8 of FIG. 6.

FIG. 9 is a view similar to FIG. 8, illustrating a disposable absorbent product in accordance with another embodiment of the invention.

FIG. 9A is a view similar to FIGS. 8 and 9, illustrating a disposable absorbent product in accordance with yet another embodiment of the invention.

FIG. 10 is a perspective view of one of the disposable absorbent products of any of FIG. 6-9 or 9A, illustrating an exemplary separation of components thereof.

FIG. 11 is a top view of a disposable absorbent product in accordance with another embodiment of the invention.

FIG. 12 is a view similar to FIG. 11, illustrating a disposable absorbent product in accordance with yet another embodiment of the invention.

FIG. 13 is a view similar to FIGS. 11 and 12, illustrating a disposable absorbent product in accordance with still another embodiment of the invention.

FIG. 14 is a view similar to FIGS. 11-13, illustrating a disposable absorbent product in accordance with another embodiment of the invention.

FIG. 15 is a view similar to FIGS. 2 and 7, illustrating a disposable absorbent product in accordance with another embodiment of the invention.

FIG. 16 is a cross-sectional view taken generally along line 16-16 of FIG. 15, illustrating the disposable absorbent product therein in a deployed position on a wearer's undergarment.

FIG. 17 is a view similar to FIG. 16, with a component of the disposable absorbent product removed.

### DETAILED DESCRIPTION OF PARTICULAR EMBODIMENTS

To the extent that any meaning or definition of a term in this written document conflicts with any meaning or definition of the term in a document incorporated by reference, the meaning or definition assigned to the term in this written document shall govern. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “joined,” “connected,” “supported,” and “coupled” and variations thereof are used broadly and encompass both direct and indirect mountings, connections, supports, and couplings. Further, “connected” and “coupled” are not restricted to physical or mechanical connections or couplings, and the term “releasably coupled” and derivatives thereof refer to a coupling of elements or components that permits their separation substantially without causing the tearing, breaking or destruction of those elements or components.

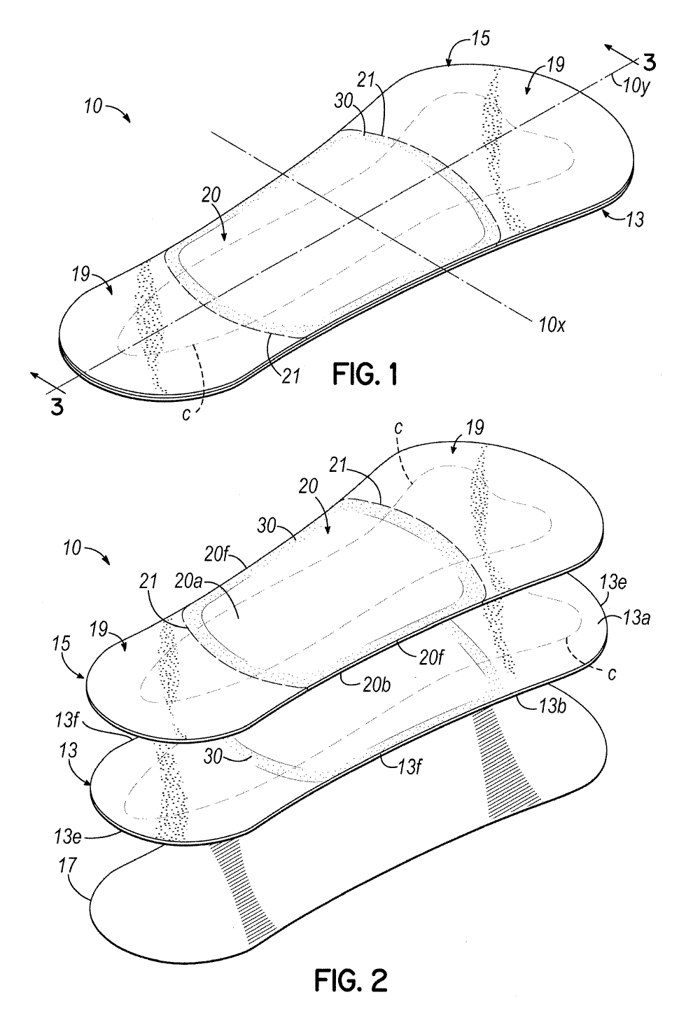

With reference to the figures, and more particularly to FIGS. 1, 2, and 3, an exemplary disposable absorbent product is illustrated, in the form of a double-liner pad 10. Pad 10 may be, for example and without limitation, a light, medium or high incontinence product that is primarily configured to be worn by a wearer so as to retain fluid e.g., urine, runny feces, secreted by the wearer of the pad 10. Alternatively or additionally, pad 10 may be a catamenial product in the form of a sanitary napkin, which is primarily configured to retain menses and related bodily secretions. Pad 10 may be intended to be a stand-alone product, designed to be directly secured to the wearer's undergarment, or may instead be designed to be worn as a removable part of a disposable adult brief, baby diaper, or training pant.

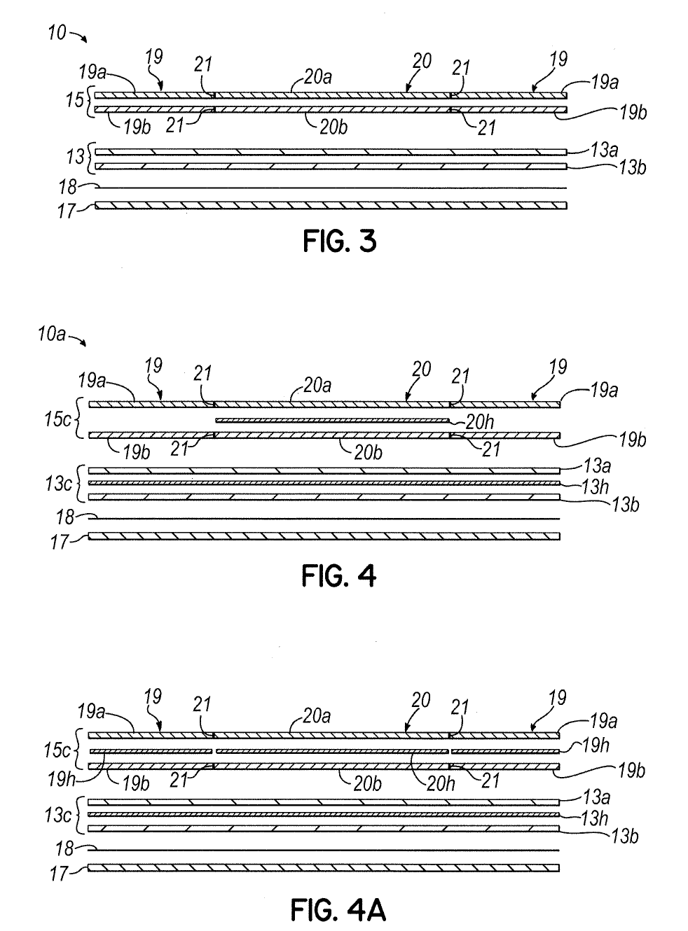

The exemplary pad 10 of FIGS. 1-3 includes first and second absorbent assemblies or liners 13, 15, overlaying one another. As more fully explained below, at least one particular component of the second absorbent liner 15 is releasably coupled to the first absorbent liner 13, so as to permit separation of that component from the rest of pad 10, when desired, without either that component or the rest of the pad 10 losing its integrity upon removal/separation. Pad 10 has a longitudinal dimension and a transverse dimension. More specifically, pad 10 extends longitudinally along a longitudinal axis 10y, and transversely along an orthogonal transverse axis 10x (FIG. 1), and has an elongate shape that is suitably chosen so as to conform to the wearer's anatomy. The exemplary embodiment of FIGS. 1-3 further includes a release liner 17, which is releasably coupled to the exposed surface of first absorbent liner 13, through a layer of adhesive 18 (FIG. 3). In use, upon removal of the release liner 17, the layer of adhesive 18 is exposed and is thereby used to releasably secure the pad 10 to the wearer's undergarment. It is understood that the layer of adhesive 18, and/or the release liner 17, may or may not form part of alternative embodiments. For example, a pad that is intended to be used as a removable part of a disposable brief (not shown) may be secured to other portions of that brief through other methods or features.

While FIG. 3 illustrates the layer of adhesive 18 as a continuous layer, generally coextensive with first absorbent liner 13, it is understood that layer of adhesive 18 may instead have any other shape and/or dimensions. Also, while the exemplary pad 10 of FIGS. 1-3 has a total of two absorbent liners 13, 15, it is contemplated that the features disclosed herein are similarly applicable to disposable absorbent products having more than two absorbent liners. For example, it is contemplated that a disposable absorbent product may have a total of 3 or 4 absorbent liners that are releasably coupled to one another, so as to permit their ready separation when desired by the wearer of the product.

In the embodiment of FIGS. 1-3, the first absorbent liner 13 and the second absorbent liner 15 have substantially the same length, although alternative embodiments are contemplated in which the lengths of the absorbent liners 13, 15 are different from one another. As used herein, the term “length” and derivatives thereof refer to the maximum dimension of the pad 10 or of any component thereof along or parallel to the longitudinal axis 10y. The second absorbent liner 15 in the illustrated embodiment includes a pair of outer subassemblies 19, and a central subassembly 20, coplanar with the outer subassemblies 19, and longitudinally located between the two outer subassemblies 19. In alternative embodiments, the central subassembly 20 is not coplanar with the outer subassemblies 19, and the outer subassemblies 19 may or may not be coplanar with one another. The outer subassemblies 19 are joined to the central subassembly 20 along respective longitudinally opposed frangible junctions 21, such as perforated tear lines, that readily permit the selective separation of the central subassembly 20 from the outer subassemblies 19, when desired by the wearer. Further, the central subassembly 20 is releasably coupled to the underlying first absorbent liner 13, as more fully explained below, to permit the selective separation of central subassembly 20 from first absorbent liner 13.

The central subassembly 20 in the embodiment of FIGS. 1-3 is, relative to the first absorbent liner 13, generally centrally positioned in the longitudinal dimension of the pad 10. In that regard, the frangible junctions 21 of second absorbent liner 15 are both respectively inboard of the adjacent longitudinally opposed end edges 13e (FIG. 2) of first absorbent liner 13. In the exemplary embodiment of FIGS. 1-3, the length of the central subassembly 20 of second absorbent liner 15 is chosen so as to minimize the use, in pad 10, of material making up central subassembly 20. In that regard, the length of the central subassembly 20 may for example be no greater than about 75% of the length of first absorbent liner 13 and/or of the length of the second absorbent liner 15, as a whole. More specifically in certain embodiments, the length of the central subassembly 20 is no greater than about 60% or even no greater than about 50% of the length of first absorbent liner 13 and/or of the length of the second absorbent liner 15, as a whole. Yet, in other embodiments, the length of the central subassembly 20 is no greater than about 40% or even no greater than about 30% of the length of first absorbent liner 13 and/or of the length of the second absorbent liner 15, as a whole. In addition, the second absorbent liner 15 may have a surface area (i.e., in the two-dimensional plane generally depicted in FIG. 1) that is no greater than about 75%, no greater than about 60%, and/or no greater than about 50% of the surface area of the first absorbent liner 13 and/or of the surface area of the second absorbent liner 15, as a whole. In specific embodiments, the surface area of the central subassembly 20 is no greater than about 40% or even no greater than about 30% of the surface area of the first absorbent liner 13 and/or of the surface area of the second absorbent liner 15, as a whole.

Further, the lateral extent i.e., the maximum dimension along or parallel to transverse axis 10x, of the central subassembly 20 of second absorbent liner 15 may be such that the central subassembly 20 and the first absorbent liner 13 are laterally (i.e., transversely) substantially coextensive. More specifically, each of the opposed lateral edges 20f (FIG. 2) of central subassembly 20 may have substantially the same transverse location as the correspondingly adjacent lateral edge 13f of the first absorbent liner 13, as in the exemplary embodiment of FIGS. 1-3. In that regard, embodiments are contemplated in which a pair of adjacent lateral edges 13f, 20f are spaced from one another by a transverse distance of about 5 mm or less, and still considered to have substantially the same transverse location. More generally, the term “coextensive,” when used to describe the relative dimensions of two components or structures of pad 10, refers to both of those components or structures having the same dimension. For example, two components are longitudinally coextensive if they have the same length (i.e., the maximum dimension along or parallel to longitudinal axis 10y), and they are laterally or transversely coextensive if they have the same width (i.e., the maximum dimension along or parallel to transverse axis 10x).

The first absorbent liner 13 of exemplary pad 10 includes a topsheet 13a, and a fluid-impervious backsheet 13b disposed opposite the topsheet 13a, such that the topsheet 13a and backsheet 13b are in an overlaying relationship with one another. In use, the topsheet 13a faces the body of the wearer, and the backsheet 13b, by contrast, faces away from the body of the wearer. The topsheet 13a may further be a fluid-pervious material, to facilitate the passage of fluid there through, particularly fluid secreted by the wearer of the pad 10. In that regard, the topsheet 13a may be made of a hydrophilic nonwoven, and may be in the form of a single, continuous layer across the length and/or width of the first absorbent liner 13, or may alternatively be in the form of two or more layers of the same material or of materials different from one another that jointly, rather than individually, span the length and/or width of the first absorbent liner 13.

The exemplary topsheet 13a also includes, as shown in FIGS. 1 and 2, a plurality of channels c, configured to direct the flow of fluid on the surface of the topsheet 13a, and which also serve as an ornamental feature designed to enhance the aesthetic appeal of pad 10. In alternative embodiments, the topsheet 13a has no channels c, or has channels in numbers and/or shapes that are different from those in the embodiment of FIGS. 1-3.

The backsheet 13b includes a fluid-impervious material, such as a hydrophobic nonwoven (e.g., spunbond nonwoven), a layer of polypropylene or polyethylene film, or a laminate combining films, nonwoven, or other materials that ultimately define a fluid-impervious laminate. Backsheet 13b may be in the form of a single, continuous layer across the length and/or width of the first absorbent liner 13, or may alternatively be in the form of two or more layers of the same material or of materials different from one another that jointly, rather than individually, span the length and/or width of the first absorbent liner 13.

With continued reference to FIGS. 1-3, the topsheet and backsheet 13a, 13b are permanently (rather than releasably) coupled to one another so as to form an integral absorbent first absorbent liner 13 that is capable of absorbing and retaining fluid secreted by the wearer of the pad 10, particularly when first absorbent liner 13 is directly exposed to the skin of the wearer. To that end, the topsheet and backsheet 13a, 13b may be coupled to one another through a suitably chosen adhesive (not shown), and/or through mechanical bonds such as heat bonds, ultrasonic bonds, CPW bonds, or other types of bonds primarily designed to melt and/or otherwise cause the permanent entanglement and/or bonding of the materials making up topsheet and backsheet 13a, 13b to one another.

The second absorbent liner 15 has a construction and materials that are similar to those of exemplary first absorbent liner 13. In that regard, the second absorbent liner 15 generally has a topsheet layer, and a fluid-impervious backsheet layer disposed opposite the topsheet layer, such that the topsheet layer and the backsheet layer are in an overlaying relationship with one another. The topsheet and backsheet layers are coupled to one another through a suitably chosen adhesive (not shown), and/or through mechanical bonds such as heat bonds, ultrasonic bonds, CPW bonds, or other types of bonds primarily designed to melt and/or otherwise cause the permanent entanglement and/or bonding of the materials making up the topsheet and backsheet layers of second absorbent liner 15 to one another. In specific embodiments, the outer and central subassemblies 19, 20 of second absorbent liner 15 are made up of a common topsheet material, and/or a common backsheet material. Yet in other embodiments, the respective topsheets and backsheets of the outer and central subassemblies 19, 20 may be different from one another. For ease of explanation and understanding, the topsheets and backsheets of the outer subassemblies 19 will be respectively designated 19a and 19b, while the topsheet and backsheet of the central subassembly 20 will be respectively designated 20a and 20b. It is understood, notwithstanding, that those chosen designations are not intended to imply that the topsheets 19a and 20a are necessarily made of different materials, or that the backsheets 19b and 20b are necessarily made of different materials.

FIGS. 4 and 4A, in which like reference numerals refer to similar features in the preceding figures, illustrate other exemplary embodiments. Referring particularly to FIG. 4, the exemplary pad 10a in that figure is similar in most respects to pad 10 (FIGS. 1-3), but includes first and second absorbent cores 13h, 20h, respectively in first and second absorbent liners 13c, 15c of that embodiment. More specifically, the first absorbent core 13h is disposed between the topsheet 13a and the backsheet 13b of first absorbent liner 13c, and the second absorbent core 20h is disposed between the topsheet 20a and the backsheet 20b of the central subassembly 20 of second absorbent liner 15c, and does not form part of the outer subassemblies 19. The absorbent cores 13h, 20h are configured to absorb and retain fluids, such as urine and/or menses, secreted by the wearer of pad 10a. Alternative embodiments are contemplated in which the outer subassemblies 19 have respective absorbent cores 19h, similar to or different from absorbent core 20h, as illustrated in FIG. 4A, or in which the absorbent core 20h extends into the outer subassemblies 19. Each of the absorbent cores 13h, 19h, 20h may have a single main absorbent structure or may instead have more than one main absorbent structure. Further, each absorbent core 13h, 19h, 20h is made up of fluff pulp or some other natural or synthetic fluid management material, and may also include a fluid storage material such as superabsorbent material (“SAP”) or some other natural or synthetic fluid storage material. In addition to the absorbent cores 13h, 19h, 20h, any or all of the first absorbent liner 13c, the outer subassemblies 19, or the central subassembly 20 may also include respective acquisition layers, adjacent the respective absorbent core 13h, 19h, 20h, primarily designed to rapidly acquire and distribute fluid received through the respective topsheet 13a, 19a, 20a.

Exemplary materials suitable for any of the absorbent cores 13h, 19h, 20h are airlaid-based materials known as “VH600.101.B6001” and “VH460.103.B6001,” both commercially available from Glatfelter Falkenhagen GmbH, of Falkenhagen, Germany. Other exemplary materials for absorbent cores 13h, 19h, 20h are foam-based materials such as those known as High Internal Phase Emulsion (HIPE) foams, of the types described, for example, in U.S. Pat. Nos. 5,387,207; 5,260,345, 5,650,222; and 5,849,805, the respective disclosures of which are hereby expressly incorporated by reference herein only for purposes of the material compositions of the HIPE foams disclosed therein.

While FIGS. 4 and 4A illustrate embodiments in which each of the absorbent liners 13c, 15c has an absorbent core 13h, 19h, 20h, alternative embodiments are contemplated in which only the first absorbent liner 13c has an absorbent core 13h, or in which only one or more of the subassemblies 19, 20 of second absorbent liner 15c has an absorbent core 19h, 20h. Similarly, it is contemplated that the absorbent core 13h in first absorbent liner 13c may have a shape, dimensions, and/or composition similar to or different from those of absorbent cores 19h, 20h, with pads incorporating such absorbent cores still falling within the scope of the present disclosure.

As discussed above, and referring again to FIGS. 1-3, the first absorbent liner 13 and the central subassembly 20 of second absorbent liner 15 are releasably coupled to one another, which permits their selective separation, when desired, by the wearer. In that regard, a low-tackiness adhesive (i.e., at room temperature) may be able to provide that type of coupling, for example by adhesively joining the lower surface of the backsheet 20b of the central subassembly 20 to the upper surface of the adjacent topsheet 13a of first absorbent liner 13. In that type of embodiment, the materials making up topsheet 13a and backsheet 20b, as well as the type of adhesive chosen to join those components to one another, may be such that the adhesive is forced to stay with the backsheet 20b upon separation of the central subassembly 20 and first absorbent liner 13 from one another. This may be desirable in some embodiments, to prevent the wearer's skin from coming in contact with the adhesive once the central subassembly 20 has been removed. For example, an adhesive may be chosen that has a bond with the backsheet 20b that is stronger than the bond with the topsheet 13a so that, upon separation, the adhesive will be carried by the central subassembly 20 being removed.

Alternatively or additionally, the first absorbent liner 13 and central subassembly 20 may be releasably coupled to one another through mechanical fasteners e.g., hook-and-loop fasteners. More specifically in one type of embodiment (not shown), the backsheet 20b or the adjacent topsheet 13a has a hook-type element, which is configured to engage a cooperating loop-type element on the confronting surface of the other of the backsheet 20b or adjacent topsheet 13a. In a contemplated variation, the backsheet 20b has on its bottom surface a hook element, which is configured to engage a surface of a fibrous material making up adjacent topsheet 13a, thereby releasably coupling the central subassembly 20 and first absorbent liner 13 to one another.

In yet another variation, illustrated in FIGS. 1 and 2, the releasable intercoupling of first absorbent liner 13 and central subassembly 20 is provided by a plurality of relatively weak pressure bonds 30 extending generally proximate and along the perimeter of central subassembly 20, and which extend at least through the thicknesses of some of the components of the first absorbent liner 13 and central subassembly 20. In the exemplary embodiment of FIGS. 1-3, the bonds 30 extend through the thickness of the topsheet 20a of central subassembly 20, making the bonds 30 visible from the upper surface of pad 10, although it is contemplated that the bonds 30 may instead not extend through the thicknesses of topsheet 20a, thereby not being visible from the upper surface of pad 10.

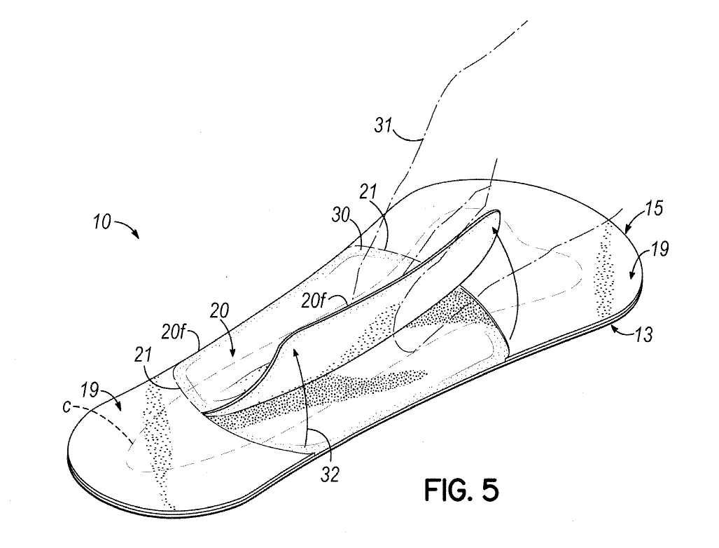

Turning now to FIG. 5, that figure illustrates an exemplary process for separating the central subassembly 20 from the rest of the pad 10. In the illustrated embodiment, the wearer's hand 31 grips the central subassembly 20 at the lateral edge 20f, and thereafter pulls the central subassembly 20 away from first absorbent liner 13, generally in the transverse direction of pad 10 (arrow 32). The pulling action of the hand 31 is effective to tear the frangible junctions 21, facilitated by the relatively stronger bond between the outer subassemblies 19 and the underlying first absorbent liner 13, which forces the outer subassemblies 19 to remain in place along with first absorbent liner 13, relative to the central subassembly 20 being removed.

In addition to the above, and while not shown in the figures, it is contemplated that the topsheet 20a of central subassembly 20 may have fluid-impervious and/or rigid substrates and/or a notch or other elements.

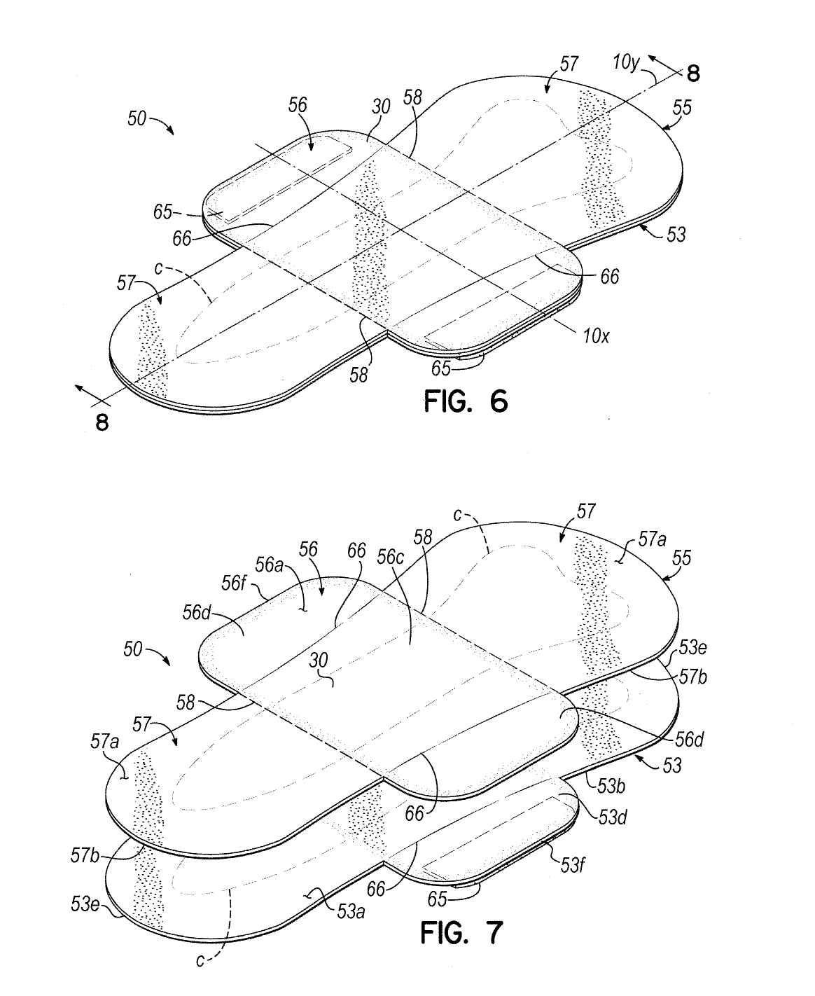

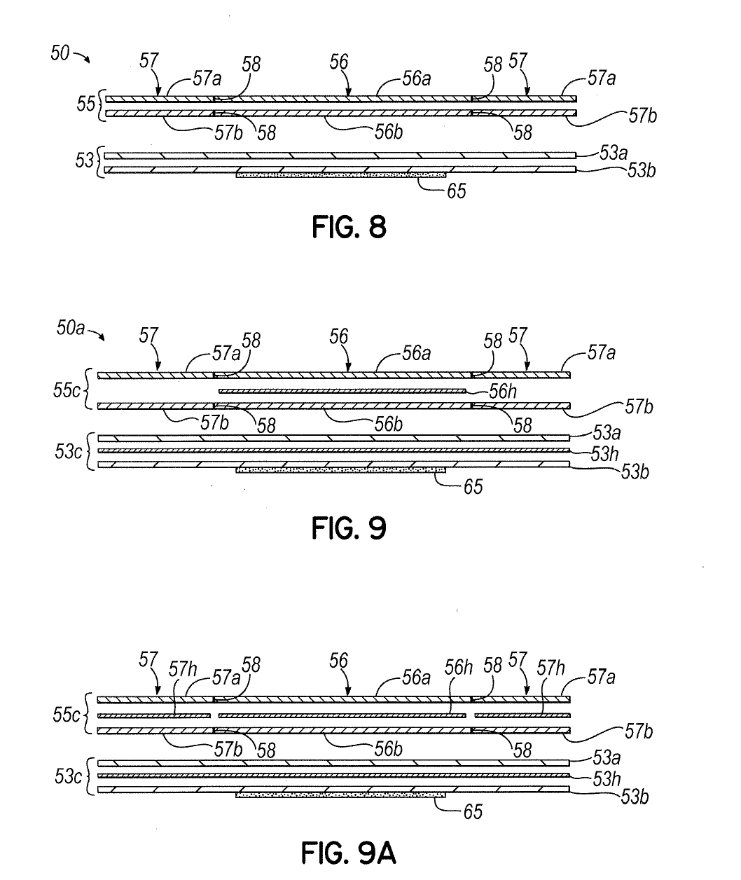

Referring now to FIGS. 6, 7, and 8, another contemplated embodiment of a double-liner pad 50 is illustrated therein. Pad 50 has a construction and is made of materials that are similar to those of pads 10, 10a of FIG. 1-4, 4a, or 5, the detailed description of which may be referred to for an understanding of the construction and materials making up pad 50 as well. For ease of understanding, like reference numerals in FIGS. 6-8 refer to similar features in the embodiments of FIGS. 1-4, 4a, and 5. Pad 50 includes a first absorbent liner 53, having a topsheet 53a and a backsheet 53b (FIG. 7) in overlaying relationship with one another, and a second absorbent liner 55 that has a shape that is similar to the shape of the first absorbent liner 53. The second absorbent liner 55 is, in turn, made up of a central subassembly 56 and a pair of outer subassemblies 57 that are coplanar with the central subassembly 56.

The central subassembly 56 is longitudinally disposed between the outer subassemblies 57 and may include materials that are different from or similar to those of the outer subassemblies 57. In that regard, the second absorbent liner 55 may for example have a common topsheet material spanning across all three subassemblies 56, 57 and/or a common backsheet material in overlaying relationship with the common topsheet material, also spanning across all three subassemblies 56, 57. Alternatively, each of the subassemblies 56, 57 may be made of respective topsheets made of materials that are different from one another. Similarly, each of the subassemblies 56, 57 may be made of respective backsheets, in overlaying relationship with the respective topsheets, made of materials that are also different from one another.

For ease of explanation and understanding, and without intending to be limiting, the topsheet and backsheet of central subassembly 56 will herein be respectively designated 56a and 56b, while the topsheets and backsheets of the outer subassemblies 57 will be designated 57a and 57b respectively. It is understood, notwithstanding, that the materials making up the topsheet 57a of one outer subassembly 57 may be similar to or different from the materials making up the topsheet 57a of the other outer subassembly 57 and further similar to or different from the topsheet 56a of the central subassembly 56. Likewise, it is also understood that the materials making up the backsheet 57b of one outer subassembly 57 may be similar to or different from the materials making up the backsheet 57b of the other outer subassembly 57 and further similar to or different from the backsheet 56b of the central subassembly 56. The central subassembly 56 is joined to the outer subassemblies 57 through respective frangible junctions 58, similar to the frangible junctions 21 of the embodiment of FIGS. 1-4, 4a, and 5. Further, the central subassembly 56 is releasably coupled to the underlying first absorbent liner 53.

Coupling between the topsheet 53a and backsheet 53b, between topsheets 56a and backsheets 56b, and/or between the topsheets 57a and respective backsheets 57b is similar to the coupling between topsheet 13a and backsheet 13b, and/or between topsheets 19a and respective backsheets 19b and/or between topsheet 20a and backsheet 20b in the embodiments of FIGS. 1-4, 4a, and 5, the detailed description of which is similarly applicable to the embodiment of FIGS. 6-8. In addition, the releasable coupling between central subassembly 56 and first absorbent liner 53 in pad 50 is similar to the releasable coupling between central subassembly 20 and first absorbent liner 13, 13c of pads 10, 10a (FIGS. 1-4, 4a, and 5), which may also be referred to for an understanding of the coupling between central subassembly 56 and first absorbent liner 53 in the embodiment of FIGS. 6-8.

With continued reference to FIGS. 6-8, the first and second absorbent liners 53, 55 are shaped so as to define a “winged” pad 50. More specifically, pad 50 has a central portion, located centrally in the transverse dimension of pad 50, and a pair of transversely opposed lateral regions that extend outwardly and protrude from the central portion, and which are foldable relative to the central portion. Once the lateral regions or “wings” of the pad 50 are folded down (with the term “down” being relative to the orientation shown in FIGS. 6-8), a pair of adhesive elements 65 on the bottom, exposed surface of backsheet 53b in the lateral regions may be used to secure the pad 50 to the underside of the wearer's undergarment. The lateral regions fold about fold lines 66 that define the border between the central portion and the lateral regions. The central subassembly 56 of second absorbent liner 55 has a central portion 56c that, jointly with a central portion 53c of the first absorbent liner 53, defines the central portion of pad 50. Further, central subassembly 56 has a pair of transversely opposed lateral regions 56d that, along with a pair of transversely opposed lateral regions 53d of first absorbent liner 53, define the lateral regions or “wings” of pad 50. The fold lines 66 define the border between the central portion 56c and lateral regions 56d of central subassembly 56, and also between the central portion 53c and lateral regions 53d of first absorbent liner 53.

The first absorbent liner 53 has longitudinally opposed, outermost end edges 53e, as well as transversely opposed, outermost lateral edges 53f (only one shown in FIG. 7). The central subassembly 56 of second absorbent liner 55 has longitudinally opposed, outermost end edges defined by the frangible junctions 58 and transversely opposed outermost lateral edges 56f. In the illustrated embodiment, the lateral edges 56f of central subassembly 56 and the respectively adjacent lateral edges 53f of first absorbent liner 53 have substantially the same location in the transverse dimension, so that the central subassembly 56 and the first absorbent liner 53 have the same lateral or transverse extent. It is contemplated, notwithstanding, that one or both of the lateral edges 56f of central subassembly 56 may be instead located transversely inboard or outboard of the respectively adjacent lateral edge(s) 53f of the first absorbent liner 53, with embodiments of that type still falling under the scope of the present disclosure. Further in the embodiment of FIGS. 6-8, the shape of the central subassembly 56 of second absorbent liner 55 is such that the central portion 56c of central subassembly 56 has the same length as do the lateral regions 56d of central subassembly 56.

In the exemplary embodiment of FIGS. 6-8, the length and/or surface area of the central subassembly 56 of second absorbent liner 55, relative to the first absorbent liner 53 and/or relative to the second absorbent liner 55, as a whole, are chosen so as to minimize the amount of material making up central subassembly 56. This would be beneficial, for example, in embodiments in which the central subassembly 56 includes relatively costly materials or materials primarily devoted to absorb or otherwise manage fluid secreted by the wearer of pad 50, while the outer subassemblies 57 do not include such materials or include those materials but in lesser amounts. More specifically, the central subassembly 56 may contain bulky absorbing materials or substrates that are relatively thick, relative to the outer subassemblies 57, which may be detrimental to the wearer's comfort. Likewise, it may be desirable to minimize the length and/or surface area of central subassembly 56 to enhance the aesthetic appeal of the pad 50 and/or to control the relative location of the frangible junctions 58 for increased comfort to the wearer.

In that regard, the length of the central subassembly 56 of second absorbent liner 55 may for example be no greater than about 75% of the length of first absorbent liner 53 and/or of the length of second absorbent liner 55, as a whole. More specifically in certain embodiments, the length of the central subassembly 56 may be no greater than about 60% or even no greater than about 50% of the length of first absorbent liner 53 and/or of the length of second absorbent liner 55, as a whole. Yet in other embodiments, the length of the central subassembly 56 may be no greater than about 40% or even no greater than about 30% of the length of first absorbent liner 53 and/or of the length of second absorbent liner 55, as a whole. In addition, the central subassembly 56 may have a surface area (i.e., in the two-dimensional planes generally depicted in FIGS. 6 and 7) that is no greater than about 75%, no greater than about 60%, and/or no greater than about 50% of the surface area of the first absorbent liner 53 and/or of the surface area of second absorbent liner 55, as a whole. In specific embodiments, the surface area of the central subassembly 56 is no greater than about 40% or even no greater than about 30% of the surface area of the first absorbent liner 53 and/or of the surface of second absorbent liner 55, as a whole.

With reference to FIG. 9, in which like reference numerals refer to similar features in the preceding figures, another exemplary embodiment of a pad 50a is illustrated. Pad 50a is similar in most respects to pad 50 (FIGS. 6-8), but includes first and second absorbent cores 53h, 56h, respectively in first and second absorbent liners 53c, 55c of that embodiment. More specifically, the first absorbent core 53h is disposed between the topsheet 53a and the backsheet 53b of first absorbent liner 53c, and the second absorbent core 56h is disposed between the topsheet 56a and the backsheet 56b of central subassembly 56. The absorbent cores 53h, 56h are configured to absorb and retain fluids, such as urine and/or menses, secreted by the wearer of pad 50a, and are similar in construction and/or materials and respective variations thereof as absorbent cores 13h, 19h, 20h of FIGS. 4 and 4A, which may be referred to for an understanding of the absorbent cores in the embodiment of FIG. 9 as well.

While FIG. 9 illustrates an embodiment in which each of the absorbent liners 53c, 55c has an absorbent core 53h, 56h, alternative embodiments are contemplated in which only the first absorbent liner 53c has an absorbent core 53h, or in which only the second absorbent liner 55c has an absorbent core 56h, or in which the absorbent core 56h is located instead between the topsheet 57a and backsheet 57b of one or both of the outer subassemblies 57. FIG. 9A illustrates another variation, in which each of the three subassemblies 56, 57 includes a respective absorbent core 56h, 57h, which may be similar to or different from one another in shape, dimensions and/or composition. Similarly, it is contemplated that the absorbent core 53h in first absorbent liner 53 may have a shape, dimensions, and/or composition different from the one or more absorbent cores 56h, 57h, with embodiments incorporating such types of absorbent cores still falling within the scope of the present disclosure.

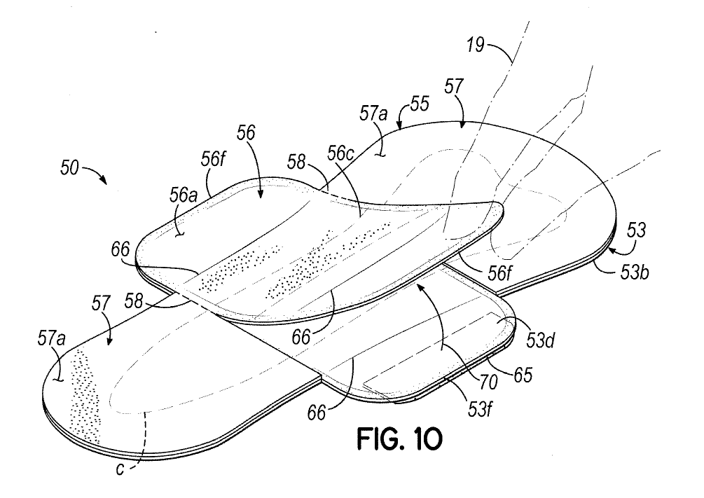

Referring now to FIG. 10, that figure illustrates an exemplary process for separation of the central subassembly 56 from the rest of the pad 50. The wearer's hand 31 is shown gripping the central subassembly 56 at one of the outermost lateral edges 56f, and thereafter pulling the central subassembly 56 away from the rest of the pad 50 (arrow 70), generally in the transverse direction of pad 50, while simultaneously holding the rest of the pad 50 down. The pulling force of the hand 31 is effective to tear the frangible junctions 58, thereby allowing separation of central subassembly 56 from the outer subassemblies 57. Further, the permanent (rather than releasable) coupling between the outer subassemblies 57 and the underlying first absorbent liner 53 is effective to maintain the outer subassemblies 57 in place, along with the underlying first absorbent assembly 53, relative to the central subassembly 56 being pulled and removed.

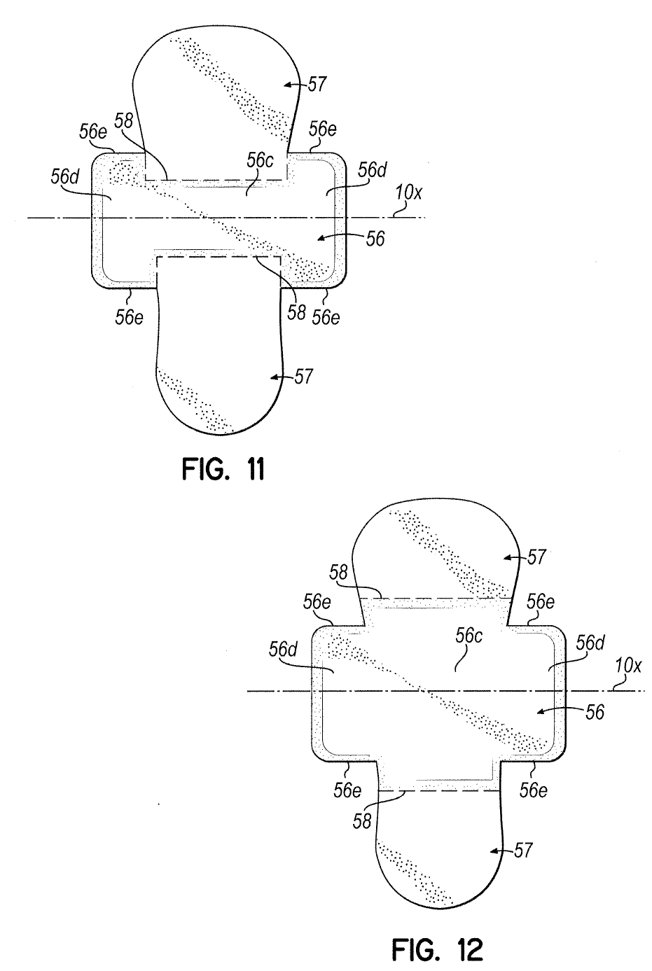

FIGS. 11, 12, 13, and 14 illustrate additional variations of the pad 50, 50a of FIGS. 6-8, which are also contemplated variations of the pads shown in FIGS. 9 and 9A. In FIGS. 11-14, like reference numerals refer to similar features in the preceding figures. FIG. 11 shows an embodiment of a pad in which both of the longitudinally opposed end edges of the central subassembly 56, defined by the frangible junctions 58, are located longitudinally inboard of the longitudinal end edges 56e of the lateral regions 56d of central subassembly 56. More specifically, each of the end edges 56e of the lateral regions 56d is located longitudinally farther from the transverse axis 10x, as axis 10x is shown in FIG. 11, relative to the longitudinal location of the frangible junctions 58. In that embodiment, accordingly, the length of the lateral regions 56d of central subassembly 56 is greater than the length of the central portion 56c of central subassembly 56.

With particular reference to FIG. 12, in the embodiment shown in that figure, both of the longitudinally opposed end edges of central portion 56c of central subassembly 56, defined by the frangible junctions 58, are located longitudinally outboard of the end edges 56e of lateral regions 56d. More specifically, in that embodiment, each of the end edges 56e of lateral regions 56d is located longitudinally closer to the transverse axis 10x, as that axis is shown in FIG. 12, relative to the longitudinal location of the frangible junctions 58. Further in that embodiment, the length of the lateral regions 56d of central subassembly 56 is less than the length of the central portion 56c of central subassembly 56.

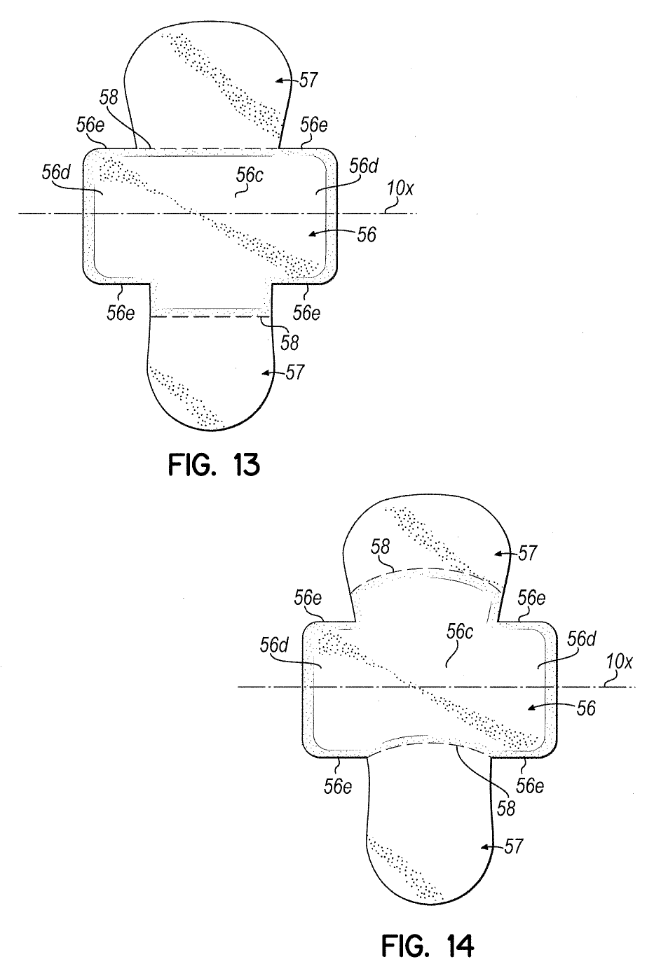

FIG. 13 illustrates another embodiment, in which the upper (relative to the orientation in that figure) end edge of central subassembly 56 is substantially linear across the entire width of central subassembly 56, such that the upper end edge of the central portion 56c, defined by the upper one of the frangible junctions 58, and the upper end edge 56e of the lateral regions 56d have a common longitudinal location i.e., they are spaced substantially by the same distance from the transverse axis 10x, as axis 10x is shown in that figure. In that embodiment, the lower (relative to the orientation in that figure), longitudinally opposed end edge of central portion 56c, defined by the lower one of the frangible junctions 58, is located longitudinally outboard of the adjacent, lower end edges 56e of lateral regions 56d. More specifically in that embodiment, the lower end edges 56e are located closer to the transverse axis 10x, as axis 10x is shown in FIG. 12, relative to the location of the lower frangible junction 58.

In the embodiment of FIG. 14, both of the longitudinally opposed end edges of central portion 56c, defined by the frangible junctions 58, have an arcuate shape. More specifically, the upper one (relative to the orientation in that figure) of the frangible junctions 58 has a concave shape, when viewed from the transverse axis 10x (as axis 10x is shown in that figure), while the lower, longitudinally opposed frangible junction 58 has a convex shape, also when viewed from the transverse axis 10x. While not shown, variations are contemplated in which one of the end edges of the central portion 56c is substantially linear while the other of the end edges of the central portion 56c is arcuate, or in which either or both of the end edges of the central portion 56c are substantially linear and oriented at an angle relative to the transverse axis 10x, rather than being parallel to axis 10x. Yet other variations are contemplated in which one or both of the end edges of central portion 56c are made up of a plurality of segments that are linear, arcuate, or which have some other shape. All of these variations are embodiments that fall within the scope and spirit of the present disclosure.

While all of the embodiments of FIGS. 1-4, 4A, 5-9, 9A, and 10-14 illustrate one removable central subassembly 20, 56 located longitudinally between two outer subassemblies 19, 57, alternative embodiments (not shown) are contemplated that include more than one removable subassembly also joined to coplanar subassemblies through at least one frangible junction. Further, other embodiments are contemplated that have one removable subassembly, similar to the exemplary central subassemblies 20, 56 of the embodiments illustrated in the figures herein, and only one other subassembly not intended for removal i.e., permanently (rather than releasably) coupled to the underlying first absorbent liner 13, 53.

For example, an embodiment is contemplated similar in most respects to the embodiment of FIGS. 1-4, and variations thereof, but which has a second absorbent liner made up of one removable subassembly extending from one longitudinal edge of the pad and releasably coupled to the underlying first absorbent liner 13, and another, non-removable subassembly extending inwardly from the opposite longitudinal edge of the pad. In that alternative embodiment, the two subassemblies are coplanar and joined to one another through a frangible junction, similar to the frangible junctions 21 in the embodiment of FIGS. 1-4.

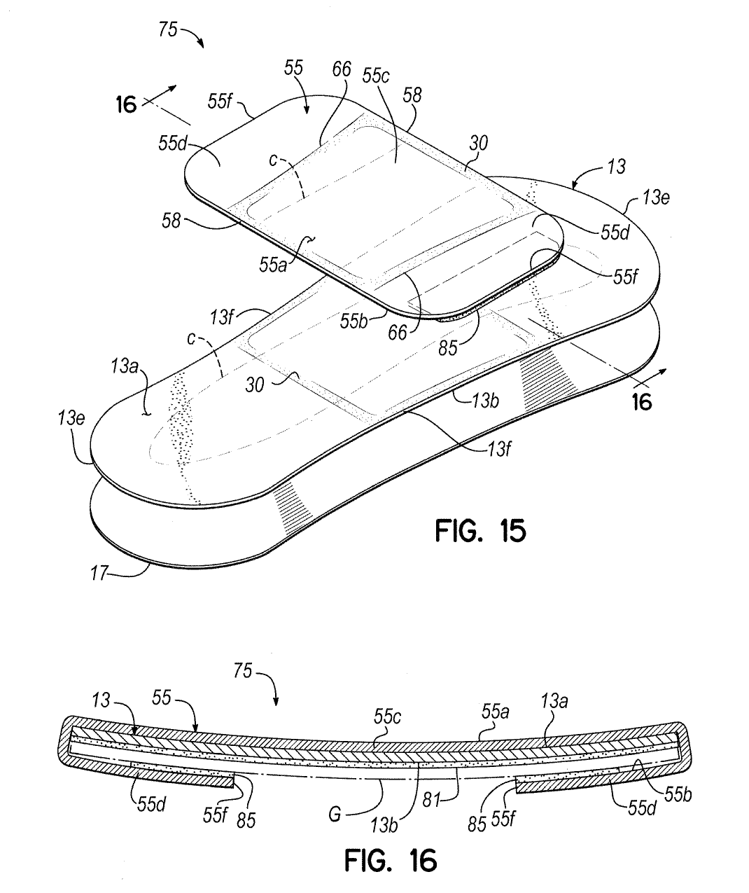

Referring now to FIGS. 15, 16, and 17, another embodiment of a disposable absorbent product in the form of a double-liner pad 75 is illustrated, similar to the disposable absorbent products of the preceding figures and contemplated variations thereof. For ease of understanding and explanation, like reference numerals in FIGS. 15-17 refer to similar features in the preceding figures. Pad 75 includes first and second absorbent liners 13 and 55, generally similar in construction and function, and contemplated variations thereof, to the like-numbered components in the embodiments of FIGS. 1-5 and 6-14, respectively. In that regard, the description of those components associated with the preceding embodiments may be referred to for an understanding of the like-numbered components in the embodiment of FIGS. 15-17 as well.

In the embodiment of FIGS. 15-17 the first, bottom absorbent liner 13 has a generally elongate shape, including a pair of opposed lateral edges 13f extending between a pair of longitudinally opposed end edges 13e. Similarly, the second, top absorbent liner 55 has a central portion 55c, and a pair of transversely opposed lateral regions 55d protruding laterally outward from central portion 55c, and longitudinally coextensive with central portion 55c in the illustrated embodiment. The lateral regions 55d may have the same construction as the central region 55c, or may have a different construction. The lateral regions 55d define wings of the pad 75, which are foldable along lines 66 that, in the illustrated exemplary embodiment, are defined by the underlying portions of the lateral edges 13f of bottom absorbent liner 13 when the lateral regions 55d are folded inwardly (FIG. 16). The fold lines 66 further define the border between central portion 55c and lateral regions 55d. The lateral regions 55d extend outwardly beyond the lateral edges 13f of the underlying absorbent liner 13, so that each of the respective outermost lateral edges 55f of the top absorbent liner 55 (i.e., at the lateral regions 55d) is transversely located distally of the respectively adjacent lateral edge 13f.

The first and second absorbent liners 13, 55 are releasably coupled to one another, in a manner similar to the manner in which the absorbent liners in the embodiments in the preceding figures are releasably coupled, so as to permit their ready separation, when desired by the wearer of the pad 75. In that regard, the absorbent liners 13, 55 may for example be coupled to one another through a plurality of relatively weak pressure bonds 30 located in the central portion 55c of second absorbent liner 55, and coupling central portion 55c to the underlying first absorbent liner 13. In the illustrated embodiment, and without intending to be limiting, the bonds 30 extend through a topsheet 55a of the second absorbent liner 55, so as to be visible from above topsheet 55a. With continued reference to FIGS. 15-17, the exemplary pad 75 has features that permit releasably securing pad 75 to the wearer's undergarment G, in use. More specifically, pad 75 includes a first fastener 81 on the bottom, exposed surface of the backsheet 13b of first absorbent liner 13. Fastener 81 may be in the form of or otherwise include an adhesive, similar to the adhesive 18 in the embodiments associated with FIGS. 3, 4, and 4A. Alternatively or additionally, fastener 81 may be in the form of or otherwise include a hook-type fastener, made up of a plurality of hooks designed for entangled engagement with a top, skin-facing surface of undergarment G. In embodiments in which the fastener 81 is or otherwise include an adhesive, pad 75 may include an optional release liner 17, similar in construction and function to the like-numbered release liner associated with the embodiment of FIGS. 1-4, 4A, and 5.

Pad 75 also includes a pair of fasteners 85, similar to the fastener 81 or contemplated variations thereof, located on the bottom surface of backsheet 55b of second absorbent liner 55, at the lateral regions 55d. The fasteners 85 may be located inboard of the outermost lateral edges 55f of lateral regions 55d, or may instead be located, as shown in the exemplary embodiment of FIG. 16, generally adjacent the outermost lateral edges 55f, at the lateral regions 55d of second absorbent liner 55. As schematically shown in FIG. 16, once the lateral regions 55d are folded inwardly (i.e., toward the longitudinal axis of pad 75), the fasteners 85 releasably engage the bottom surface of undergarment G, thereby further securing pad 75 to undergarment G.

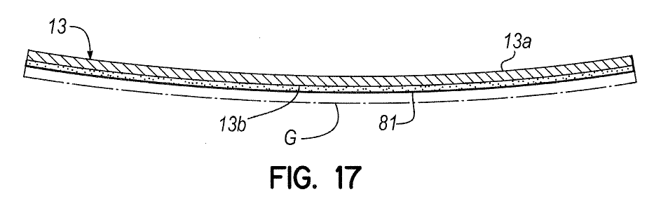

Second absorbent liner 55 may be separated from the rest of pad 75, when desired by the wearer, in ways similar to the separation of the absorbent liners in the embodiments of FIGS. 1-14. Separation of the second absorbent liner 55 from the first absorbent liner 13, in that regard, includes disengagement (e.g., detachment) of the fasteners 85 from undergarment G. As schematically shown in FIG. 17, once second absorbent liner 55 is removed, the first fastener 81 remains in place, thereby maintaining pad 75 releasably secured to undergarment G. While FIGS. 15-17 illustrate an exemplary second absorbent liner 55 that is generally rectangular in shape, alternative embodiments are contemplated in which second absorbent liner 55 has another suitably chosen shape. For example, and without limitation, second absorbent liner 55 could have a shape similar to any of the exemplary central subassemblies 56 in the embodiments illustrated in FIGS. 11-14.

Yet other contemplated variations include the second absorbent liner 55 of FIGS. 15-17 having two or more subassemblies joined to one another through frangible junctions, as in the embodiment of FIG. 6-9, 9A, and 10, for example. The advantages of the exemplary construction and relative dimensions of the absorbent liners 13, 55 in the embodiment of FIGS. 15-17 and contemplated variations thereof are similar to those discussed in connection with the embodiments of FIGS. 1-14, resulting in minimization of the use of materials making up at least part of the second absorbent liner 55 for increased comfort to the wearer and/or a more efficient use of those materials.

From the above disclosure of the general principles of the present invention and the preceding detailed description of exemplary embodiments, those skilled in the art will readily comprehend the various modifications to which this invention is susceptible. Accordingly, this invention is intended to be limited only by the scope of the following claims and equivalents thereof.

1. A disposable absorbent product having a longitudinal dimension and a transverse dimension orthogonal to the longitudinal dimension, the disposable absorbent product comprising:
a first absorbent assembly including a first topsheet and a fluid-impervious first backsheet in overlaying relationship with one another, said first backsheet and said first topsheet being respectively configured to face away from and toward a wearer of the disposable absorbent product during use, said first absorbent assembly having a pair of lateral edges; and
a second absorbent assembly overlaying said first absorbent assembly, and being releasably coupled to said first absorbent assembly, said second absorbent assembly including a second topsheet configured to face toward the wearer of the disposable absorbent product during use and a second backsheet adjacent said first topsheet and in overlaying relationship with said second topsheet, said second absorbent assembly having a central portion and a pair of opposed lateral regions protruding transversely from said central portion so as to define a pair of foldable wings of the disposable absorbent product, wherein said lateral regions of said second absorbent assembly have respective opposed outer lateral edges, each of said outer lateral edges being located distally from an adjacent one of said lateral edges of said first absorbent assembly.

2. The disposable absorbent product of claim 1, further comprising:
a first fastener on an exposed surface of said first backsheet, adapted to secure the disposable absorbent product to a garment worn by the wearer thereof; and
a second fastener in said lateral regions of said second absorbent assembly, adapted to secure the disposable absorbent product to the garment worn by the wearer thereof.

3. The disposable absorbent product of claim 2, wherein at least one of said first fastener or said second fastener includes an adhesive.

4. The disposable absorbent product of claim 2, wherein at least one of said first fastener or said second fastener includes a plurality of hooks adapted for entangled engagement with the garment worn by the wearer of the disposable absorbent product.

5. The disposable absorbent product of claim 2, wherein said second fastener includes a pair of opposed adhesive elements, each inboard of a respective adjacent one of said outer lateral edges at said lateral regions.

6. The disposable absorbent product of claim 2, wherein said second fastener is disposed on said second backsheet.

7. The disposable absorbent product of claim 1, wherein said lateral regions are foldable along respective fold lines, each fold line defining a border between said central portion and said lateral regions of said second absorbent assembly.

8. The disposable absorbent product of claim 7, wherein said fold lines are defined by said lateral edges of said first absorbent assembly when said lateral regions are folded.

9. The disposable absorbent product of claim 1, wherein said first and second absorbent assemblies are releasably coupled to one another through a plurality of bonds located in said central portion of said second absorbent assembly.

10. The disposable absorbent product of claim 9, wherein said bonds extend through said second topsheet of said second absorbent assembly.

11. The disposable absorbent product of claim 1, wherein said first topsheet is fluid pervious and said second backsheet is fluid impervious.

12. The disposable absorbent product of claim 1, wherein at least one of said first or second absorbent assemblies has an absorbent core disposed between said first topsheet and said first backsheet, or between said second topsheet and said second backsheet, respectively.

13. A disposable absorbent product having a longitudinal dimension and a transverse dimension orthogonal to the longitudinal dimension, the disposable absorbent product comprising:
a first absorbent assembly including a first topsheet and a fluid-impervious first backsheet in overlaying relationship with one another, said first backsheet and said first topsheet being respectively configured to face away from and toward a wearer of the disposable absorbent product during use, said first absorbent assembly having a pair of lateral edges;
a second absorbent assembly overlaying said first absorbent assembly, and being releasably coupled to said first absorbent assembly, said second absorbent assembly including a second topsheet configured to face toward the wearer of the disposable absorbent product during use and a second backsheet adjacent said first topsheet and in overlaying relationship with said second topsheet, said second absorbent assembly having a central portion and a pair of opposed lateral regions protruding transversely from said central portion so as to define a pair of foldable wings of the disposable absorbent product;
a first adhesive on an exposed surface of said first backsheet, adapted to secure the disposable absorbent product to a garment worn by the wearer thereof; and
a second adhesive on said second backsheet, in said lateral regions of said second absorbent assembly, adapted to secure the disposable absorbent product to the garment worn by the wearer thereof, wherein said lateral regions of said second absorbent assembly have respective opposed outer lateral edges, each of said outer lateral edges being located distally from an adjacent one of said lateral edges of said first absorbent assembly.

14. A disposable absorbent product having a longitudinal dimension and a transverse dimension orthogonal to the longitudinal dimension, the disposable absorbent product comprising:
a first absorbent assembly including a first topsheet and a fluid-impervious first backsheet in overlaying relationship with one another, said first backsheet and said first topsheet being respectively configured to face away from and toward a wearer of the disposable absorbent product during use; and
a second absorbent assembly overlaying said first absorbent assembly, and being releasably coupled to said first absorbent assembly, said second absorbent assembly including a second topsheet configured to face toward the wearer of the disposable absorbent product during use and a second backsheet adjacent said first topsheet and in overlaying relationship with said second topsheet, said second absorbent assembly having a length no greater than about 75% of a length of said first absorbent assembly.

15. The disposable absorbent product of claim 14, wherein said second absorbent assembly has a length no greater than about 50% of the length of said first absorbent assembly.

16. The disposable absorbent product of claim 14, wherein said second absorbent assembly has a central portion and a pair of opposed lateral regions protruding transversely from said central portion so as to define a pair of foldable wings of the disposable absorbent product.

17. The disposable absorbent product of claim 16, wherein said first absorbent assembly has a central portion and a pair of opposed lateral regions protruding transversely from said central portion of said first absorbent assembly, so as to further define said pair of foldable wings of the disposable absorbent product.

18. The disposable absorbent product of claim 16, wherein said central portion of said second absorbent assembly is longitudinally coextensive with said lateral regions thereof.

19. The disposable absorbent product of claim 16, wherein said central portion of said second absorbent assembly has a pair of longitudinally opposed end edges, said end edges being substantially linear.

20. The disposable absorbent product of claim 16, wherein said central portion of said second absorbent assembly has a pair of longitudinally opposed end edges, at least one of said end edges being arcuate.
